(19) United States
(12) Patent Application Publication
(10) Pub. No.: US 2012/0178994 A1
(43) Pub. Date: Jul. 12, 2012

(54) APPARATUS AND METHOD FOR ASSISTING IN THE DELIVERY OF MEDICAL INSTRUMENTS INTO BODY ORGANS

(76) Inventor: Drew Schembre, Seattle, WA (US)

(21) Appl. No.: 13/344,407

(22) Filed: Jan. 5, 2012

Related U.S. Application Data
(60) Provisional application No. 61/431,752, filed on Jan. 11, 2011.

(51) Int. Cl. A61B 1/00

(52) U.S. Cl. 600/115

(57) ABSTRACT

An apparatus assists in the delivery of a medical instrument to within a body organ of a body wherein the body organ has an inner wall. The apparatus includes an inflatable structure arranged to attach to the medical instrument with a minimum profile when deflated to permit the medical instrument and the attached deflated structure to be inserted into the body organ. The inflatable structure is inflatable to the inflatable structure to enlarge and grip the inner wall of the organ while permitting movement of the medical instrument relative to the inflatable structure and the body organ. The inflatable structure is deflatable to permit the inflatable structure to be removed from the body organ.

### PRIORITY CLAIM

The present application claims the benefit of copending U.S. Provisional Patent Application Ser. No. 61/431,752, filed Jan. 11, 2011, which application is incorporated herein by reference in its entirety.

### BACKGROUND OF THE INVENTION

The present invention generally relates to an apparatus and method that assists in the delivery of medical instruments into body organs. For example, embodiments of the invention are directed to delivering a colonoscope into the colon of a patient's body. Other applications will become readily apparent.

Colonoscopy is a medical procedure during which a video camera mounted on a controllable, flexible narrow shaft is inserted through the rectum to the most proximal portion of the colon. This enables physicians to visualize the lining of the colon in order to identify abnormal and potentially pre-cancerous tissue and either biopsy it or remove it. Colonoscopy has become a common and widely available diagnostic and therapeutic tool and more recently, the major screening tool for detecting and preventing colon cancer by facilitating the removal of pre-cancerous polyps. While colonoscopy is now widely available, there remains broad variability in how well it is performed, how long it takes, how much anesthesia and other medical resources are required to perform the procedure. There also remains broad variability in the opinion of how safely it can be done.

Part of the reason for this variability of opinion is the variability in patient anatomy and physician training. But a large part of the problem lies with the devices themselves used to perform the procedure. The technique and tools of colonoscopy remain largely unchanged from when the procedure was first described in 1967 as basically pushing a flexible tube through the floppy colon. The average colon is about 6 feet in length and except for a fibrous attachment at the splenic flexure (about ⅓ of the way up the colon) and at the anus, it moves freely in the abdomen, tethered by a web of tissue on one surface, called the mesentery. The lower or distal ¼ of the colon, called the sigmoid colon, is an “S” shaped tube that is redundant and prone to looping.

The primary instrument used in performing colonoscopies is the colonoscope. A colonoscope is an elongated flexible tubular instrument having a plurality of working channels extending there through. Unfortunately, passing a colonoscope through the colon invariably causes stretching of the colon as the sigmoid colon twists and loops. This causes looping of the colon that reduces the effective distance the scope can be passed as force applied to the handle of the scope is lost as the scope bows and bends. This stretching also causes the majority of the discomfort associated with colonoscopy. Most looping occurs in the sigmoid colon but can also occur in the mid colon (transverse colon) or in the right colon in patients with extremely long colons. Much of the skill in performing colonoscopy is involved in reducing these loops, shortening the colon by retracting the scope and pleating bowel over the scope to enable scope passage more proximally. Frequently, external pressure or moving the patient is required to keep loops from forming or to hold sleeved bowel on the scope.

Similar looping can occasionally occur in the stomach and small intestine during upper endoscopies. This is usually less of a problem as the esophagus is straight and fixed in place in the chest. The stomach is directly below the esophagus and there is rarely much stretching or looping as the upper endoscope is passed into the duodenum. Problems arise in the setting of previous surgery where a loop of bowel may have been introduced between the esophagus and the stomach or bile or pancreatic ducts. In these situations, looping of bowel can often make it impossible to pass a scope through the mouth to the desired endpoint.

Overtube devices have been used successfully in the small bowel and colon to splint the bowel in order to decrease looping. However, these overtubes can be awkward to use, expensive to purchase and rigid. This can cause additional discomfort and some risk of tissue damage. The main purpose of an overtube is to direct vector forces and reduce looping and bowing rather than actually reduce the loops of bowel in an effort to shorten the bowel.

Hence there is a need in the art for an improved procedure and apparatus to enable the delivery of medical instruments into body organs, such as the delivery of a colonoscope into the colon. The present invention addresses these and other issues.

### SUMMARY OF THE INVENTION

According to an embodiment of the invention, an apparatus is provided for assisting in the delivery of a medical instrument to within a body organ of a body, wherein the body organ has an inner wall. The apparatus comprises an inflatable structure arranged to attach to the medical instrument with a minimum profile when deflated to permit the medical instrument and the attached deflated structure to be inserted into the body organ. The inflatable structure is inflatable causing the inflatable structure to enlarge and grip the inner wall of the organ while permitting movement of the medical instrument relative to the inflatable structure and the body organ. The inflatable structure is also deflatable to permit the inflatable structure to be removed from the body organ.

The apparatus may further comprise a breakable suture. The breakable suture releasably attaches the inflatable structure to the medical instrument and releases the inflatable structure from the medical instrument when the inflatable structure is inflated to permit movement of the medical instrument relative to the inflatable structure and the body organ.

The apparatus may further comprise a tether for fixing the inflatable member to the body. The tether may be an inflation tether.

The apparatus may further comprise a pull-cord that attaches the inflatable structure to the medical instrument. The inflatable structure may comprise a balloon. The balloon may be ring-shaped. The ring-shaped balloon may have a center opening that receives the medical instrument.

The balloon may alternatively be a spiral balloon that wraps around the medical instrument. As a further alternative, the balloon may be asymmetrically shaped having an inflatable lateral aspect that enlarges and grips the inner wall of the organ and another aspect opposite the inflatable lateral aspect that is non-inflatable. Another aspect of the asymmetrically shaped balloon may be a concave surface arranged to be adjacent the medical instrument. As a still further alternative, the inflatable structure may comprises a plurality of balloons, such as a pair of balloons.

According to further embodiments, another apparatus is provided for assisting in the delivery of a medical instrument to within a body organ of a body, wherein the body organ has an inner wall. The apparatus comprises at least one balloon arranged to attach to the medical instrument. The at least one balloon and medical instrument have a minimum profile when deflated to permit the medical instrument and the attached deflated at least one balloon to be inserted into the body organ. The at least one balloon is inflatable causing the at least one balloon to enlarge and grip the inner wall of the organ while permitting movement of the medical instrument relative to the at least one balloon and the body organ. The at least one balloon is also deflatable to permit the inflatable structure to be removed from the body organ. The apparatus further comprises a tether to have a first end connected to the at least one balloon and a second end attachable to an external portion of the body or other external anchor.

In a further embodiment, a method of introducing a medical instrument into an organ of a body, wherein the organ has an inner wall includes the steps of releasably attaching an inflatable structure to the instrument with the inflatable structure initially being in a deflated state, inserting the instrument with the deflated inflatable structure into the body organ to a desired depth, inflating the inflatable structure to cause the inflatable structure to contact and grip the organ inner wall, withdrawing the medical instrument and inflatable structure to foreshorten the organ, and advancing the medical instrument further into the organ while maintaining the foreshortening of the organ with the inflatable structure. the inflatable structure is releasably attached to the medical instrument after the medical instrument has been inserted into the body organ while the inflatable structure is in a deflated state and wherein the inflatable structure is advanced into the body organ along the medical instrument before being inflated.

The inflatable structure may be releasably attached to the medical instrument after the medical instrument has been inserted into the body organ while the inflatable structure is in a deflated state. The inflatable structure may then be advanced into the body organ along the medical instrument before being inflated.

### BRIEF DESCRIPTION OF THE DRAWINGS

The features of the present invention which are believed to be novel are set forth with particularity in the appended claims. The various described embodiments of the invention, together with representative features and advantages thereof, may best be understood by making reference to the following description taken in conjunction with the accompanying drawings, in the several figures of which like reference numerals identify identical elements, and wherein:

FIGS. 1-3 are partial perspective views of a first embodiment of the invention wherein a balloon is releasably attached to a medical instrument by a breakable suture;

FIGS. 4 and 5 are partial perspective views of further embodiments wherein balloons are attached to medical instruments by pull-cords;

FIGS. 6-9 are partial perspective views of a still further embodiment of the invention wherein an inflatable structure includes a flexible low profile shaft and a series of loose rings attach the inflatable structure and low profile shaft to a medical instrument;

FIGS. 10-12 are partial perspective views of another embodiment of the invention wherein the inflatable structure is a ring-shaped balloon;

FIG. 13 is a partial perspective view of another embodiment of the invention wherein the inflatable structure is an asymmetrically shaped balloon and wherein the balloon has not yet been inflated;

FIG. 14 is a sectional view of the balloon of FIG. 13;

FIG. 15 is another partial perspective view of the embodiment of FIG. 13 after the balloon has been inflated;

FIG. 16 is a sectional view of the balloon of FIG. 15;

FIGS. 17-19 are partial perspective views of another embodiment of the invention wherein the inflatable structure is a spiral-shaped balloon;

FIGS. 20 and 21 are partial perspective views of another embodiment of the invention wherein the inflatable structure is a pair of balloons;

FIG. 22 is a partial perspective view of the embodiment of FIGS. 20 and 21 in a deflated state and attached to a colonoscope;

FIG. 23 is another partial perspective view of the embodiment of FIGS. 20 and 21 in a inflated state;

FIGS. 24-30 are partial perspective views of the embodiment of FIGS. 20-23 and showing a series of operative steps in delivering the embodiment of FIGS. 20-23 into a colon according to an embodiment of the invention;

FIG. 31 is a sectional view of a colon having therein a colonoscope and a pair of balloons attached thereto and in a deflated state; and

FIG. 32 is a sectional view of a colon having therein a colonoscope and a pair of balloons attached thereto and in an inflated state.

### DETAILED DESCRIPTION OF THE DRAWINGS

Referring now to FIGS. 1-3 which are partial perspective views of a first embodiment of the invention wherein a balloon is releasably attached to a medical instrument by a breakable suture. To that end, the embodiment 40 of FIGS. 1-3 includes an inflatable structure 42, a breakable suture 44, and a tether 46. The inflatable structure may be, for example, a balloon. The balloon 42 is attached to a medical instrument 50, such as a colonoscope. As may be seen in FIG. 1, the breakable suture forms a loop around the colonoscope 50 to releasably secure the balloon to the colonoscope 50 with the balloon in a deflated condition.

The tether 46 may be formed of a tubing 48. The tether 46 thus performs the dual function of inflating the balloon and maintaining the balloon in a fixed position after the balloon has been inflated. As a result, the tether 46 may be referred to as an inflation tether.

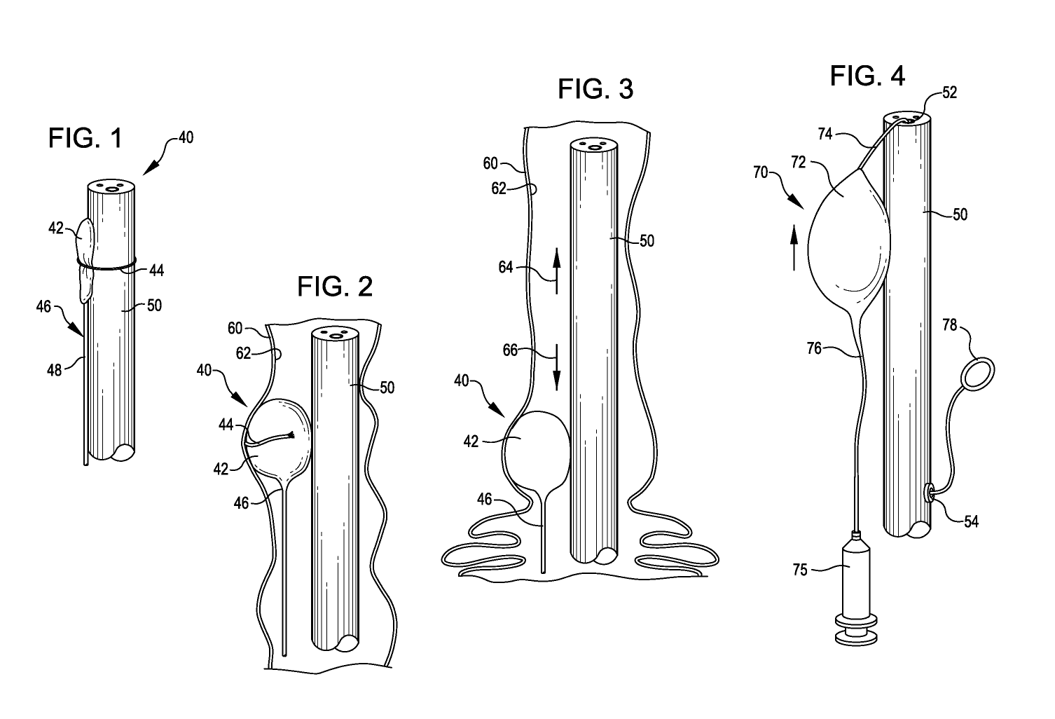

In using the embodiment of FIGS. 1-3, the endoscope 50 with the attached deflated balloon 46 is inserted to a desired depth within a body organ having an inner wall. The balloon 42 may now be inflated using the inflation tether 46. This may be best seen in FIG. 2. Here it may be noted that the colonoscope 50 has been inserted into the body organ 60, such as a colon having an inner wall 62. The colonoscope has been inserted to a desired depth within the colon 60 and the balloon 42 has been inflated. The balloon 42 may be inflated with, for example, either a pre-measured syringe (not shown) or a pressure-controlled automatic inflation device (not shown) of the type well known in the art. The balloon may be inflated with air or another gas or with a fluid, such as saline. Inflating the balloon breaks the suture 44 which previously attached the balloon to the colonoscope. The balloon 42 is now able to grip the inner wall 62 of the colon 60 when it is inflated.

The colonoscope 50 and balloon 42 may now be withdrawn several centimeters together. The combined gripping of the colon inner wall 62 as the colonoscope 50 and balloon 42 are withdrawn causes foreshortening the colon 60. FIG. 3 illustrates the colon 60 being foreshortened. The inflation tether 46 may then be fixed externally with an adhesive dressing to the thigh of the patient. The colonoscope 50 is then advanced beyond the balloon 42 indicated by arrows 64 and 66 as in a normal colonoscopy, with the advantage that the bowel is now less likely to loop and bow. After reaching the desired depth with the colonoscope 50, the balloon 42 can be deflated and withdrawn either before the colonoscope is withdrawn or while the colonoscope is being removed.

Referring now to FIG. 4, it shows another embodiment including an inflatable structure, such as balloon 72, an inflation tether 76, and a long-pull cord 74. The long pull-cord 74 serves to releasably attach the balloon 72 to the colonoscope 50. The pull-cord is attached to the tip of the balloon and passes up to the tip of the colonoscope 50, through and down a working channel 52, and out through an access port 54 of the colonoscope 50 by its scope handle. The pull-cord 74 terminates in a grip ring 78.

With the embodiment 70 of FIG. 4, the pull-cord 74 holds the balloon 72 against the colonoscope 50. Once the balloon has been inflated with the syringe 75, the balloon grips the inner wall of the colon 72 and it along with the colonoscope 50 may be withdrawn to foreshorten the colon (not shown). The inflation tether 76 may be secured to the patient's thigh and the colonoscope 50 may be advanced further into the colon. The balloon 72 may now be deflated and drawn up by the pull-cord to the tip of the colonoscope. As the deflated balloon is pulled up the colonoscope, the colonoscope may be used to exert a slight pressure against the colon wall to maintain the foreshortening of the colon. The balloon may now be re-inflated at a point deeper in the bowel. The balloon and colonoscope may once again be withdrawn to further foreshorten the bowel. This may be continued until the desire location in the bowel has been reached by the tip of the colonoscope. This allows the scope and balloon to “inchworm” though the bowel.

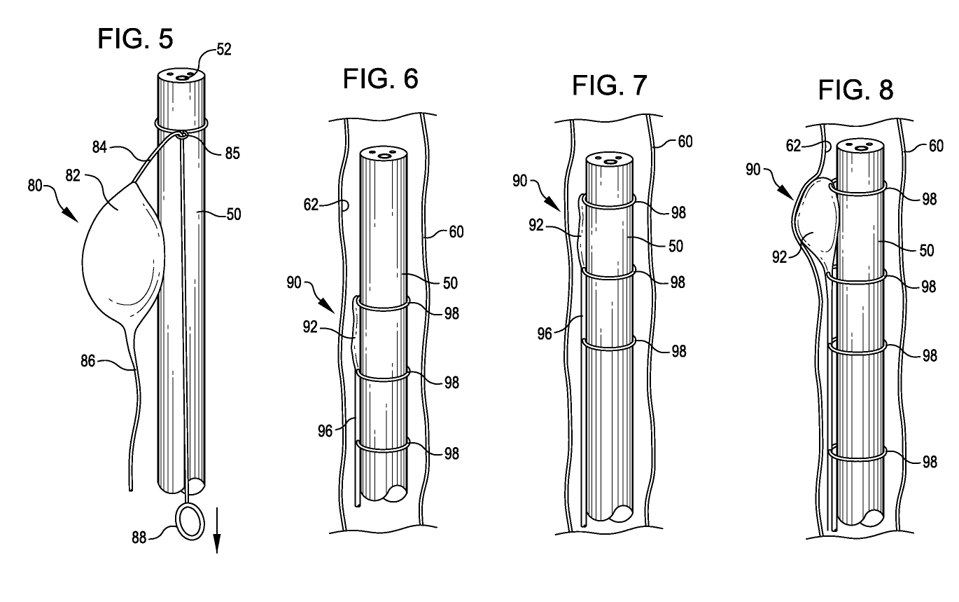

FIG. 5 shows a similar embodiment 80. The embodiment 80 includes an inflatable structure, such as balloon 82, an inflation tether 86, and a long-pull cord 84. As in the previous embodiment, the long pull-cord 84 serves to releasably attach the balloon 82 to the colonoscope 50. As in the previous embodiment, the pull-cord 84 is attached to the tip of the balloon 82. Here however, the pull-cord 84 passes through an external ring 85 at the tip of the colonoscope 50, and extends down alongside the colonoscope 50 to terminate in a grip ring 88.

In the embodiment 80 of FIG. 5, the pull-cord 84 holds the balloon 82 against the colonoscope 50. Once the balloon has been inflated, the balloon 82 grips the inner wall of the colon and it, along with the colonoscope 50, may be withdrawn to foreshorten the colon (not shown). The inflation tether 86 may be secured to the patient's hip and the endoscope 50 may be advanced further into the colon. The balloon 82 may now be deflated and drawn up by the pull-cord 84 to the tip of the colonoscope. As the deflated balloon is pulled up the colonoscope, the colonoscope may be used to exert a slight pressure against the colon wall to maintain the foreshortening of the colon. The balloon may now be re-inflated at a point deeper in the bowel. The balloon and colonoscope may once again be withdrawn to further foreshorten the bowel. This may be continued until the desire location in the bowel has been reached by the tip of the colonoscope. This, as in the previous embodiment, allows the scope and balloon to “inchworm” though the bowel.

FIGS. 6-9 are partial perspective views of a still further embodiment 90 of the invention wherein an inflatable structure includes a flexible low profile shaft and a series of loose rings attach the inflatable structure and low profile shaft to a medical instrument such as, for example, a colonoscope 50. More specifically, as may be seen in FIG. 6, the embodiment 90 includes a balloon 92, a flexible low profile shaft 96, and a plurality of spaced apart rings 98. The low profile shaft would be joined to an inflation/traction tether like the inflation tethers shown in previous embodiments. The inflation tether is not shown in this embodiment so as to not unduly complicate the figures. The balloon 92 and the shaft 96 are attached to the colonoscope 50 by the plurality rings that loosely encircle, but do not bind, the colonoscope 50.

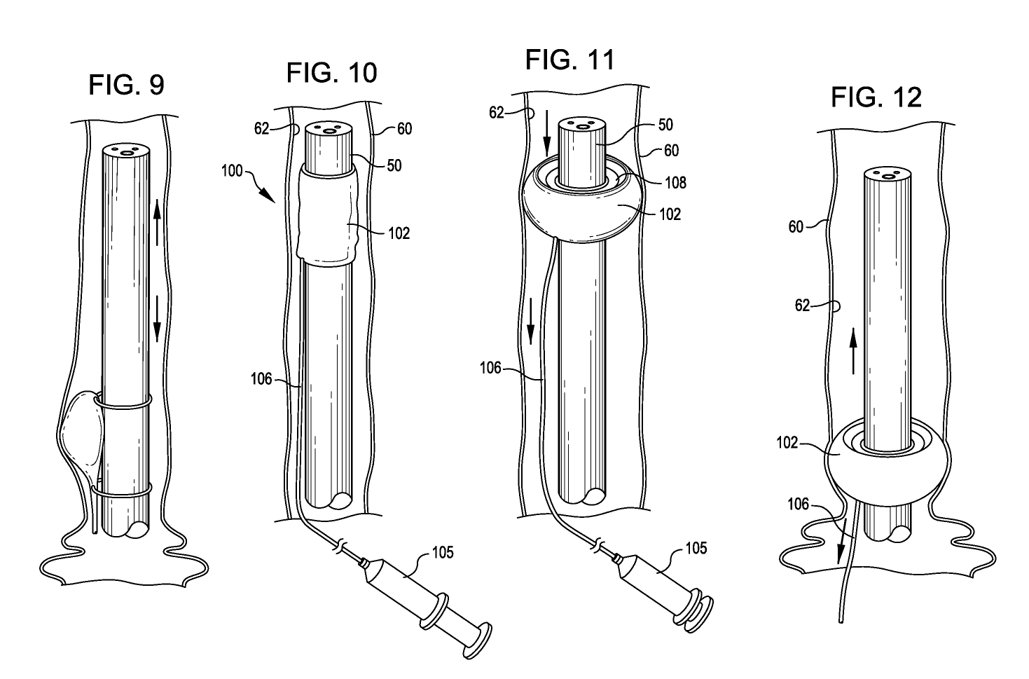

As seen in FIG. 7, after successful colon shortening with inflation of the balloon 92 to grip the inner wall 62 of the colon 60 and withdrawal of the balloon 92 and the colonoscope 50, the balloon 92 may be deflated and advanced along the colonoscope 50 by pushing the shaft 96. After the balloon 92 reaches its next desired position on the colonoscope 50, the balloon 92 may be inflated again to grip the inner wall 62 of the colon 60. The balloon 92 and colonoscope 50 may once again be withdrawn, a desired amount. The inflation tether (not shown) may once again be secured to the patient to provide traction and the endoscope may be advanced to its next position as shown in FIG. 9. The foregoing may be repeated for sleeving more bowel and allowing the colonoscope to be advanced further into the colon. As may be appreciated by those having skill in the art, the flexible shaft may be employed in combination with a pull-cord as well.

FIGS. 10-12 are partial perspective views of another embodiment 100 of the invention wherein the inflatable structure is a ring-shaped balloon 102. FIG. 10 shows the balloon 102 in its deflated state and has an inner diameter slightly less than the outer diameter of the colonoscope 50. The balloon will also have enough elasticity to secure it by friction just behind the bending rubber at the tip of the colonoscope. Preferably, the deflated balloon has a low profile of only a few millimeters so as to not impede normal passage of the colonoscope as illustrated in FIG. 10. As may be seen in FIG. 11, when the balloon 102 is inflated with syringe 105 through the inflation tether 106, the balloon 102 will attain a donut or ring shape with an outer diameter of several centimeters to provide lateral pressure and tension on the colon wall 62 to grip the colon 60. The inflation of the balloon 102 may have 2 stages. At stage 1, the balloon 102 will grip the colon but remain attached to the scope 50 as illustrated in FIG. 11. This permits the balloon 102 and the colonoscope 50 to be withdrawn together to foreshorten the colon. At stage 2, a fixed web or fold 108 in the ring will break and the inner diameter of the ring will expand to several millimeters greater than the outer diameter of the endoscope and thereby allow the colonoscope to move freely through the ring as illustrated in FIG. 12 to perform the colonoscopy. At this time, the inflation tether may be secured to the patient. When deflated, the balloon 102 will retract and enable the balloon to be withdrawn over the endoscope. The internal pressure of the balloon will not be high enough to perforate the colon, tear the lining or compromise blood flow to the colon mucosa. Ridges may be added to the balloon to enhance friction.

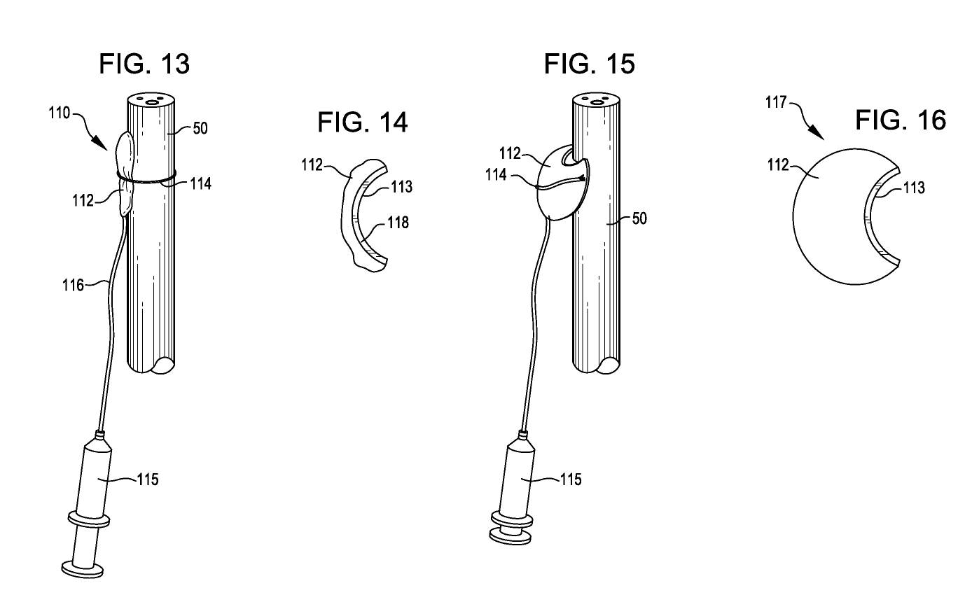

FIGS. 13-16 illustrate yet another embodiment 110 wherein the balloon is asymmetrically shaped. FIG. 13 is a partial perspective view of the asymmetrically shaped balloon 112 before it is inflated. As may be seen in the sectional view of FIG. 14, prior to inflation, the balloon has a shallow C-shape. It has a substantially rigid base 118 having a concave surface 113 for contacting the colonoscope. The traction balloon inflates asymmetrically, expanding laterally from the endoscope toward the colon wall when inflated by a syringe 115, for example, through an inflation tether 116. FIGS. 15 and 16 illustrate the balloon 112 in its inflated state. The side of the balloon 112 adjacent to the colonoscope 50 that forms the base does not inflate but instead maintains its concave shape. The lateral aspect 117 of the balloon inflates in a hemi-sphere shape to anchor the colon. This allows the colon to be anchored with less friction on the endoscope.

The balloon 112 is releasably attached to the colonoscope by a breakable suture. When the balloon 112 is inflated, the suture breaks (FIG. 15). At this point, the balloon grips the colon. The colonoscope 50 and the balloon 112 are then withdrawn a desired amount to foreshorten the colon. The inflation tether is then secured to the patient and the colonoscope is used to perform the colonoscopy. When it is desired to remove the balloon 112, the balloon is simply deflated and withdrawn.

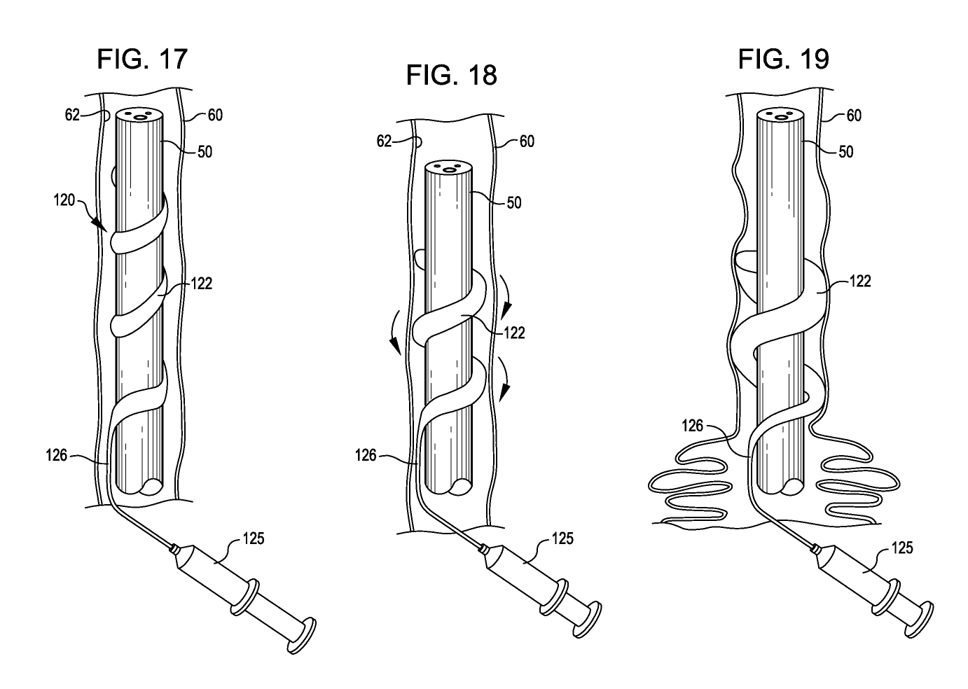

FIGS. 17-19 are partial perspective views of another embodiment 120 of the invention wherein the inflatable structure is a spiral-shaped balloon 122. The spiral-shaped balloon 122 is formed of a non-elastic material in a spiral configuration. It is formed to initially wrap snugly around an colonoscope 50 in the deflated state as shown in FIG. 17. The balloon 122 has a low-profile to pass with the colonoscope into the colon. At a point where looping occurs, the spiral balloon 122 is inflated by a syringe 125 or pressure-sensing pump through an inflation tether. As the spiral balloon expands during inflation, it increases its inner diameter. It eventually frees itself from the colonoscope and exerts pressure on the colon inner wall 62 as seen in FIG. 18. Once inflated, the balloon and colonscope may be withdrawn together to foreshorten the colon 60 as shown in FIG. 19. Traction may then be applied to the spiral balloon by securing the inflation tether to the patient as previously described. The colonoscope 50 may then be advanced further into the colon 60 without looping.

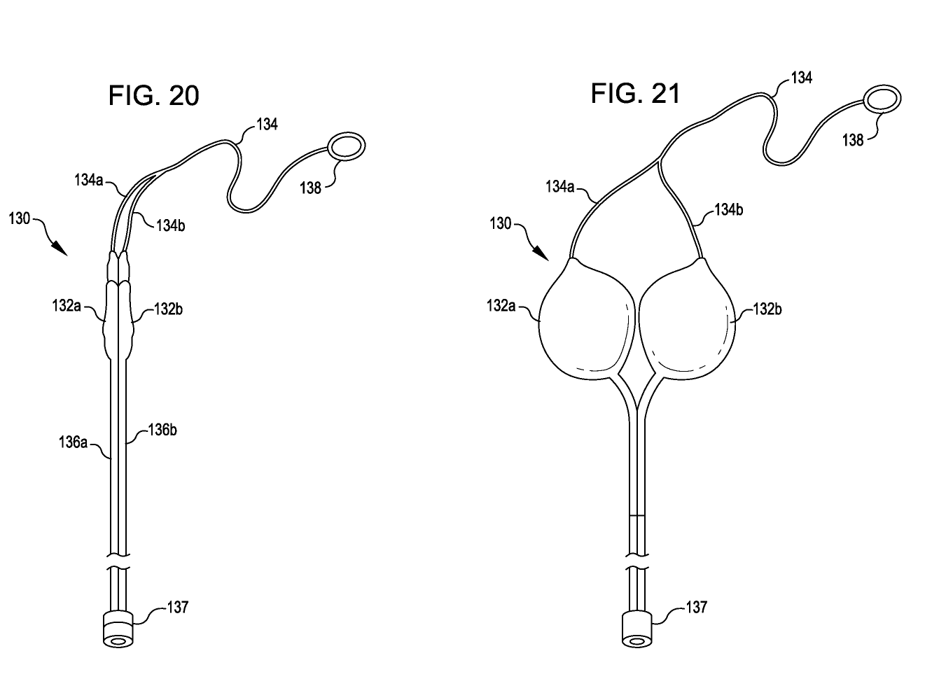

FIGS. 20-23 are partial perspective views of another embodiment 130 of the invention wherein the inflatable structure is a pair of balloons 132a and 132b.

In this embodiment, two (or more) balloons 132a and 132b are may attached side by side and inflated together to create a traction anchor that is not circular but instead, a figure “8” in cross section. This creates lateral force and friction on the bowel wall but does not completely fill or conform to the bowel lumen, leaving a gap through which and colonoscope may pass with less friction and less pressure on the colon wall. Balloons could be filled by a common inflation channel or separately.

To this end, it may be seen in FIG. 20 that two balloons 132a and 132b are side by side. Each balloon has its own inflation tether 136a and 136b respectively and pull cord 134a and 134b which extend from the tips of the balloons and join as a single cord 134 that terminates in a grip ring 138. The balloons 132a and 132b have a low profile. The pull cords are preferably of high-strength. The pull cord 134 may extend through a working channel of the colonoscope or alongside the colonoscope. A Luer-lock 137 may be provided at the proximal end for inflation and as a traction anchor. In the inflated state, the twin balloons occupy a much greater space than would be occupied by a single balloon. FIG. 21 shows the balloons 132a and 132b in an inflated state.

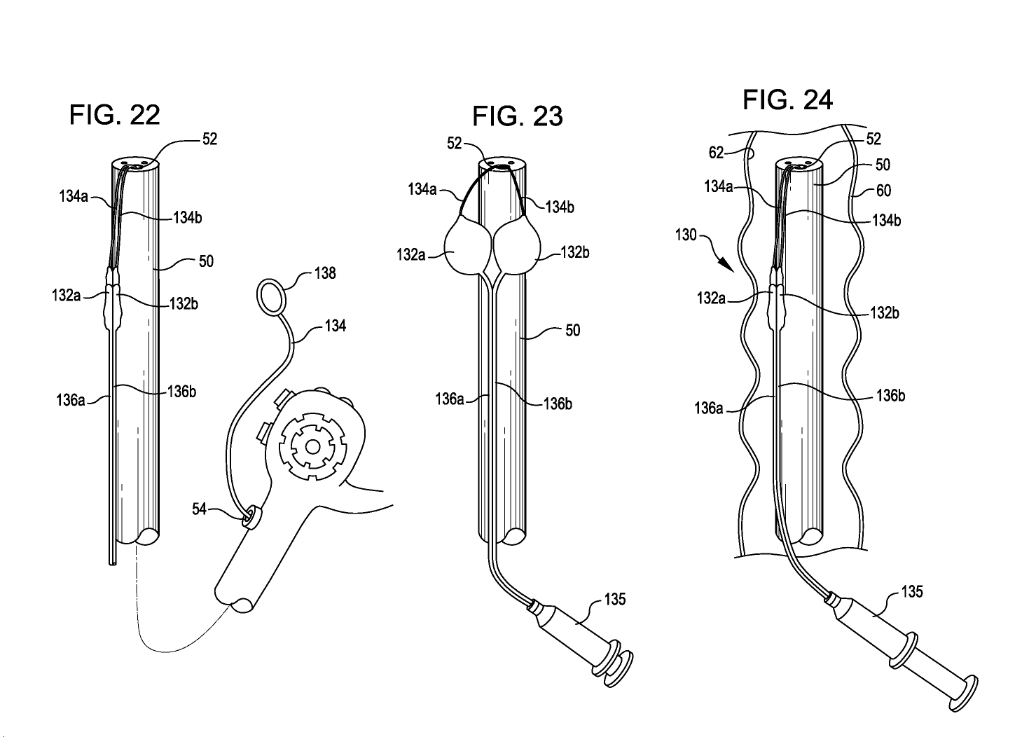

FIG. 22 shows the pull cords 134a and 134b extending through a working channel 52 of the colonoscope 50. The pull cords join into a single cord 134 that exits the colonoscope 50 at an access port 54 of the colonoscope 50. FIG. 23 shows the balloons 132a and 132b after having been inflated by syringe 135 through the inflation tethers 136a and 136b and after having been pulled to the tip of the colonoscope 50.

FIGS. 24-30 are partial perspective views of the embodiment of FIGS. 20-23 and showing a series of operative steps in delivering the embodiment of FIGS. 20-23 into a colon according to an embodiment of the invention. FIG. 24 shows the colonoscope 50 in the left colon with the balloons 132a and 132b in a deflated state. The colonoscope 50 and the attached twin balloons 132a and 132b are passed into the colon 60 under direct visualization, as a normal with colonoscopes. The deflated twin balloons 132a and 132b have a low profile and do not pose much resistance to scope passage.

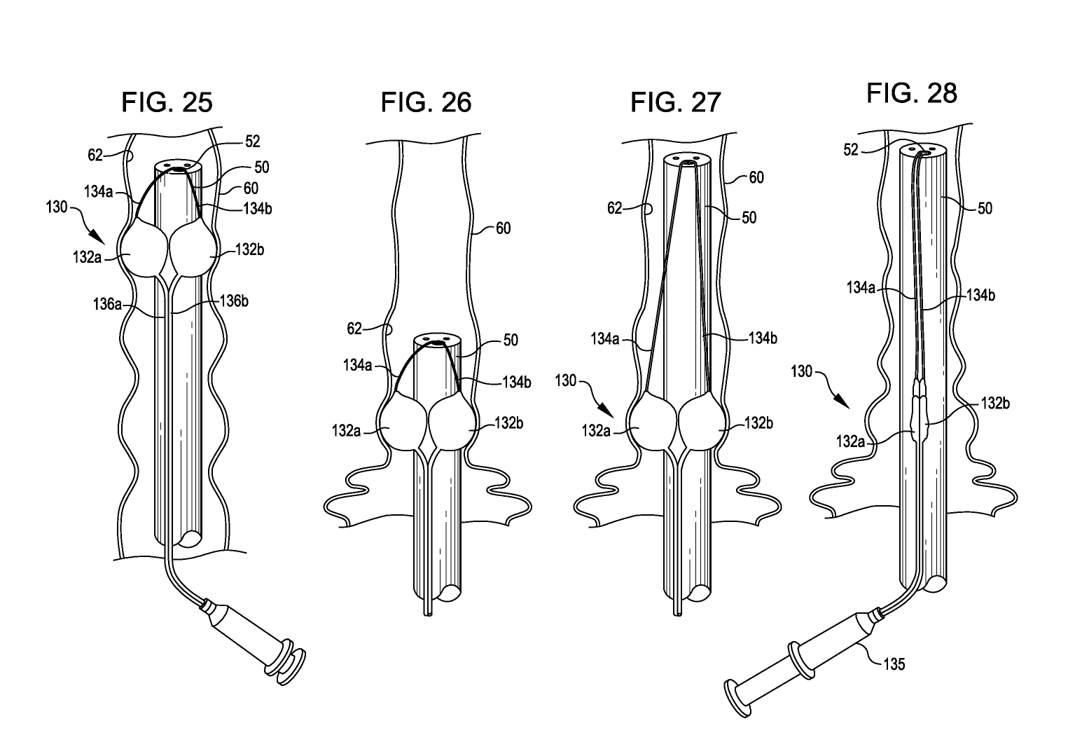

At a point where the colonoscope begins to loop or looses 1:1 advancement, the twin balloons 132a and 132b are inflated (FIG. 25). This provides lateral contact with the colon wall 62. Muscle rings (haustral folds) provide natural narrowings in the colon which conform to the expanded balloons 132a and 132b and help hold them in place when the colonoscope and twin balloons are pulled proximally together (FIG. 26). This pleats and shortens the colon 60 as it is collapsed against the lower pelvic structures and straightens out the sharply angulated sigmoid colon and hold it is place. The inflation tubing/anchor 136a, 136b is then taped to the patient's buttock or thigh to keep the colon pleated. This helps avoid creating too much tension on the twin balloons if the patient moves, as the taped anchor will move with him or her and will move in tandem with the patient.

With the twin balloons inflated, the tether cords 134a and 134b are freed and the colonoscope 50 is pushed proximally through the now shortened and straightened colon (FIG. 27) without the sigmoid colon following the colonoscope and un-pleating because it is being held in place by the traction forces of the twin balloons.

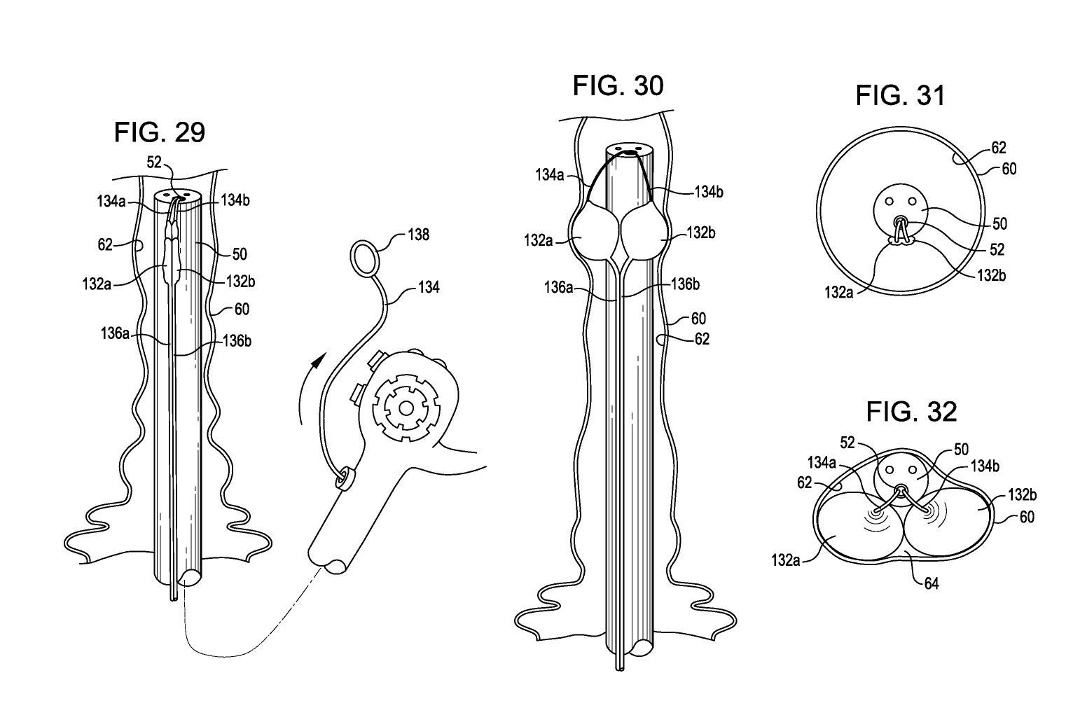

When the colonoscope 50 begins to loop again, the twin balloons 132a and 132b are deflated (FIG. 28) and pulled up the colonoscope 50 to a position near the tip by pulling on the ringed pull cord 134 near the colonoscope handle (FIG. 29). With the balloons 132a and 132b deflated, it is unlikely that many pleats of colon will fall off the shaft of the colonoscope and the colon is likely to remain in a shortened position.

With the twin balloons 132a and 132b again near the tip of the colonoscope 50, they can be inflated again, the scope can be withdrawn a short distance and even more pleats can be formed (FIG. 30). This can be continued throughout the colon. The same process, but with less inflation and distention could be performed in the small intestine.

FIG. 31 shows a cross sectional view of the colon 60. The colonoscope has a low profile, and the balloons 132a and 132b are deflated. With the twin balloons 132a and 132b inflated in FIG. 32, the colon 60 distends to an ellipse but with two gaps 64 and 66 between the balloons. This becomes an ideal space to pass the colonoscope through as it offers less resistance than would be encountered with a single balloon filling the colon lumen.

When the cecum or most proximal target is reached, the twin balloons are deflated and removed simply by pulling on the inflation tether. The colonoscope could then be withdrawn in the usual fashion.

As can be appreciated from the foregoing, traction balloons are provided that can be one of a variety of designs whereby the inflated balloon creates static friction on the colon or intestinal wall allowing the organ to be shortened in an accordion-like manner and held in place by a tether that is fixed externally while the colonoscope is advanced beyond the balloon in to a now shorter, non-looping organ. There may be a variety of designs for the balloons, their mechanism of attachment to the colonoscope, the release from the colonoscope and whether or not they may be advanced secondarily after deflation or simply remain in one position until removed.

As may be appreciated from the forgoing, the embodiments disclosed herein may find uses other than specifically disclosed herein. For example, the embodiment of FIGS. 6-9 may be useful as a “rescue” apparatus as for example, during a standard colonoscopy procedure where the colonoscope has already been inserted into the colon and the physician finds that the colon is too long and loopy to safely or comfortably get the scope all the way around in the colon. Rather than completely remove the colonoscope and start the procedure over, the apparatus of FIG. 6 could be applied to the colonoscope 50 by attaching the rings 98 to the colonoscope. To that end, the rings may be formed from strings that can be tied to the colonoscope. The balloon 92 could then be advanced up the already inserted colonoscope on the shaft 96 while being loosely confined by the rings 98. When the balloon reaches the desired position, it may be inflated as previously described and used to further foreshorten the colon as the balloon 92 and colonoscope 50 are withdrawn as previously described.

Hence, while particular embodiments have been shown and described, it is to be understood that modifications may be made. Hence, the descriptions and illustrations herein are therefore intended to encompass all such changes and modifications.

What is claimed is:

1. An apparatus for assisting in the delivery of a medical instrument to within a body organ of a body, the body organ having an inner wall, the apparatus comprising an inflatable structure arranged to attach to the medical instrument with a minimum profile when deflated to permit the medical instrument and the attached deflated structure to be inserted into the body organ, the inflatable structure being inflatable causing the inflatable structure to enlarge and grip the inner wall of the organ while permitting movement of the medical instrument relative to the inflatable structure and the body organ, and the inflatable structure being deflatable to permit the inflatable structure to be removed from the body organ.

2. The apparatus of claim 1, further comprising a breakable suture that releasably attaches the inflatable structure to the medical instrument and releases the inflatable structure from the medical instrument when the inflatable structure is inflated to permit movement of the medical instrument relative to the inflatable structure and the body organ.

3. The apparatus of claim 1, further comprising a tether for fixing the inflatable member to the body.

4. The apparatus of claim 3, wherein the tether is an inflation tether.

5. The apparatus of claim 1, further comprising a pull-cord that attaches the inflatable structure to the medical instrument.

6. The apparatus of claim 1, wherein the inflatable structure comprises a balloon.

7. The apparatus of claim 6, wherein the balloon is ring-shaped.

8. The apparatus of claim 7, wherein the ring-shaped balloon has a center opening that receives the medical instrument.

9. The apparatus of claim 6 wherein the balloon is a spiral balloon that wraps around the medical instrument.

10. The apparatus of claim 6, wherein the balloon is asymmetrically shaped having an inflatable lateral aspect that enlarges and grips the inner wall of the organ and another aspect opposite the inflatable lateral aspect that is non-inflatable.

11. The apparatus of claim 10, wherein the another aspect of the asymmetrically shaped balloon has a concave surface arranged to be adjacent the medical instrument.

12. The apparatus of claim 1, wherein the inflatable structure comprises a plurality of balloons.

13. The apparatus of claim 12, wherein the plurality of balloons is a pair of balloons.

14. An apparatus for assisting in the delivery of a medical instrument to within a body organ of a body, the body organ having an inner wall, the apparatus comprising:
at least one balloon arranged to attach to the medical instrument, the at least one balloon and medical instrument having a minimum profile when deflated to permit the medical instrument and the attached deflated at least one balloon to be inserted into the body organ, the at least one balloon being inflatable causing the at least one balloon to enlarge and grip the inner wall of the organ while permitting movement of the medical instrument relative to the at least one balloon and the body organ, and the at least one balloon being deflatable to permit the inflatable structure to be removed from the body organ; and
a tether have a first end connected to the at least one balloon and a second end attachable to the body.

15. A method of introducing a medical instrument into an organ of a body, the organ having an inner wall, the method comprising:
releasably attaching an inflatable structure to the instrument, the inflatable structure initially being in a deflated state;
inserting the instrument and the deflated inflatable structure into the body organ to a desired depth;
inflating the inflatable structure to cause the inflatable structure to contact and grip the organ inner wall;
withdrawing the medical instrument and inflatable structure to foreshorten the organ; and
advancing the medical instrument further into the organ while maintaining the foreshortening of the organ with the inflatable structure.

16. The method of claim 15, wherein the inflatable structure is releasably attached to the medical instrument after the medical instrument has been inserted into the body organ while the inflatable structure is in a deflated state and wherein the inflatable structure is advanced into the body organ along the medical instrument before being inflated.
